(19) United States
(12) Patent Application Publication
(10) Pub. No.: US 2011/0214671 A1
(43) Pub. Date: Sep. 8, 2011

(54) INHALER ASSEMBLY

(75) Inventors: Calvin London, Victoria (AU); Helle Funch Nielsen, Hoersholm (DK)

(73) Assignee: PULMOVET APS, Hoersholm (DK)

(21) Appl. No.: 12/739,618

(22) PCT Filed: Oct. 23, 2008
(86) PCT No.: PCT/AU2008/001561

§ 371 Date: May 27, 2011

(30) Foreign Application Priority Data
Oct. 24, 2007 (AU) 2007905813

(51) Int. Cl. A61M 15/00

(52) U.S. Cl. 128/203.12

(57) ABSTRACT

The present invention provides an inhaler assembly for providing medications in aerosol form to animals, especially equines. The inhaler assembly comprises a holder adapted to retain at least two diffusion canisters containing either substantially the same or different drug formulations. The canister holder has an outlet in communication with the inlet of a spacer. The spacer, in turn, has an outlet in communication with a mask such that, in use, medication from at least one of the canisters can be dispensed into the spacer for inhalation in aerosol form via the mask.

### FIELD OF THE INVENTION

The present invention relates to an inhaler assembly for the delivery of inhalation medications to animals.

### BACKGROUND ART

The following discussion of the background art is intended to facilitate an understanding of the present invention only. The discussion is not an acknowledgement or admission that any of the material referred to is or was part of the common general knowledge as at the priority date of the application.

Many different animals suffer from respiratory disorders which remain difficult to treat because of the inability to provide effective medication. These include horses, which are dependent on a large supply of oxygen to fuel their large muscles, particularly those horses associated with performance (e.g. racing, cross-country, show jumping). It is recognised that between 25-92% of stabled horses have some form of airway inflammation, which can lead to loss of performance or at the least an impaired quality of life. Furthermore, it has also been reported that respiratory problems are the third leading cause of lost work days in the horse population.

Other animals too can also suffer from respiratory disorders. Cats, particularly aging cats, typically have respiratory or airway inflammation as a result of ingesting large amounts of fur when grooming themselves. Swine can suffer from the fatal disease PRRS (Porcine Reproductive and Respiratory Reproductive Syndrome) and cattle can suffer respiratory disorders often associated with stress and changes to their surroundings, such as movement from one feedlot herd to another.

In many cases, while the symptoms are obvious, treatment is often lacking due to the difficulty of administering appropriate medications which may include, in some instances, the delivery of two or three combination drugs. Although some treatments for respiratory disorders exist, as is the case in treating heaves in horses, these are usually in the form of oral medications. Oral medications are in certain instances considered to be particularly inappropriate for treatment of disorders such as heaves, as large concentrations are required to overcome the losses caused by systematic administration and the development of unwanted effects (side-effects) resulting from the high concentrations of drugs administered. The delivery of specific medications to the site of the disorder has remained relatively underdeveloped in modern veterinary medicine due to the absence of an effective and easy-to-use delivery system.

The present invention aims to provide an inhaler assembly which provides an easier and more effective method of administering such medication or at least provides a useful alternative to existing apparatus.

### DISCLOSURE OF THE INVENTION

The present invention provides an inhaler assembly comprising a holder adapted to retain at least two diffusion canisters containing either substantially the same or different drug formulations, the canister holder having an outlet in communication with the inlet of a spacer, the spacer having an outlet in communication with a mask wherein, in use, medication from at least one of the canisters can be dispensed into the spacer for inhalation via the mask.

Preferably, the holder is adapted to retain at least three diffusion canisters.

The canister holder preferably includes an elongated holding portion having a separate receiving portion for receiving and retaining each canister. The holding portion preferably includes an open first end and a closed second end having an outlet formation extending therefrom substantially perpendicularly to the holding portion.

The receiving portions are preferably joined to each other to hold the canisters side by side and parallel to each other in use. The holding portions preferably include cutout portions therein which form windows.

The canisters preferably contain an aerosol propellant and medication to release a fixed dose of medication in aerosol form when actuated. The, or each, canister preferably includes a metering dose valve with an actuating stem. The closed second end of the holding portion preferably holds a discharge nozzle for each canister which mates with the respective metering valve and actuating stem for releasing aerosolised medication through an outlet of the outlet formation.

The canister holder preferably includes a closing cap for placing over the canisters in the holding portion, the closing cap having an end face having an actuator access aperture for each canister. The cap is preferably detachably attached to the holding portion.

The canister holder is preferably attachable to an actuator having an actuating arm for actuating the, or each, canister. The actuator preferably includes a retaining portion for receiving and retaining the canister holder outlet formation and wherein the actuator arm is attached to the retaining portion by connectors. The retaining portion preferably includes a panel having a substantially central opening and a flange formation adapted for slidably receiving and retaining the outlet formation of the canister holder, wherein the outlet formation outlet is substantially in line with the panel opening.

The actuator arm preferably includes a first surface having grip formations and an opposed second surface from which a protrusion extends generally towards the retaining portion wherein, in use, the actuator arm extends over the closing cap and the protrusion extends into one of the cap apertures.

The spacer preferably includes a substantially closed first end face, side walls extending therefrom, and an open second end which together define a chamber therewithin, wherein the end face includes an opening in communication with the canister holder outlet and the second end is in communication with the mask. The first end face preferably includes a recess which is adapted for releasably receiving and retaining the actuator, wherein the recess includes an aperture centrally formed therein which corresponds to the aperture in the retaining portion.

The design of the spacer has been engineered to permit the maximum distribution of a drug into the free space of the chamber. Activation of the canisters releases the desired drug into the conical shaped diffuser. The cloud of micro particles is generated under pressure, said pressure being relieved when the inhaler assembly of the invention is applied to an animal and the animal breathes and hence inhales a volume of air through the diffuser. The volume of air inhaled is in the proportion of approximately 10:1 (clean air: air containing drug) facilitating the delivery of the drug into the airways of the animal.

The end face preferably includes at least one one-way valve for allowing air into the chamber but substantially preventing the exit of air from the chamber. Each one-way valve preferably includes an opening which is closable by a flap made from a flexible but resilient material.

The mask preferably includes a body dimensioned and shaped to cover a single nostril of the nose of an animal as desired, such as a horse. The body preferably includes a first open end shaped to substantially form a seal around the animal's nostril, and a second end attached to the spacer second end.

The mask can be adapted to the body dimensions of any large animal (including, but not limited to, horses, cattle, sheep, camels, donkeys, ponies), small animals (including, but not limited to, cats, dogs and birds) and humans, both adult and children.

The assembly preferably includes a one-way valve between the spacer and the mask.

The assembly preferably includes canisters containing medication which are to be administered in sequence or in combination with each other. These may include, for example, bronchodilators that facilitate the relaxation of the trachea through which humans and animals breathe, corticosteroids that reduce the inflammatory reaction to airway disorders, anti-mucolytic agents such as cromoglycate, antibiotics that reduce the microbial load associated with an infection and other forms of medication aimed at enhancing the quality of breathing or reducing in the short or longer term the severity of the disorder.

The present invention in another aspect provides an assembly comprising the inhaler assembly as above having at least two diffusion canisters received and retained therein.

The present invention in another aspect provides a method of administering at least two types of inhalation medication in sequence to an animal using the above inhaler assembly, the method comprising:
depressing a first medication canister to release the desired dose of first medication into the spacer chamber;
applying the mask to cover a single nostril of the animal while the second nostril is manually occluded by the operator thereby forcing the animal to breathe through the nostril covered by the mask such that aerosolised medication in the chamber is inhaled by the animal;
repeating the above steps with the other medication canisters as required.

The present invention in another aspect provides a method of administering at least two types of inhalation medication in combination to an animal using the above inhaler assembly, the method comprising:
depressing the medication canisters to release the desired dose of medication from each canister into the spacer chamber;
applying the mask to cover a single nostril of the animal while the second nostril is manually occluded by the operator thereby forcing the animal to breathe through the nostril covered by the mask such that aerosolised combination of medication in the chamber is inhaled by the animal.

The medication may be one or more medications used to treat a condition selected from any one or more of: heaves, Chronic Obstructive Pulmonary Disorder (COPD), Inflammatory Airway Disease (IAD), Recurrent Airway Obstruction (RAO), and influenza.

According to a further aspect of the invention, there is provided the use of the inhaler assembly as described herein in a method of treating any one or more of: heaves, Chronic Obstructive Pulmonary Disorder (COPD), Inflammatory Airway Disease (IAD), Recurrent Airway Obstruction (RAO), and influenza.

### BRIEF DESCRIPTION OF THE DRAWINGS

Preferred forms of the present invention will now be described by way of examples only, with reference to the accompanying drawings, wherein: FIG. 1 shows two canisters and a canister holder for the inhaler assembly according to a preferred embodiment of the present invention;

FIG. 2 shows the two canisters inserted into the canister holder;

FIG. 3 shows the closing cap for the canister holder being attached thereto to retain the canisters within the holder;

FIG. 4 shows the assembled canister holder of FIG. 3 being attached to an actuator for the inhaler assembly;

FIG. 5 shows the canister holder attached to the actuator;

FIG. 6 shows the actuator and canister holder assembly of FIG. 5 being attached to a first half of a spacer of the inhaler assembly;

FIG. 7 shows the actuator and canister holder assembly attached to the first half of the spacer; and

FIG. 8 shows the assembled inhaler assembly where the first half of the spacer in the assembly of FIG. 7 is attached to a second half of the spacer and a mask for a horse.

### BEST MODE(S) FOR CARRYING OUT THE INVENTION

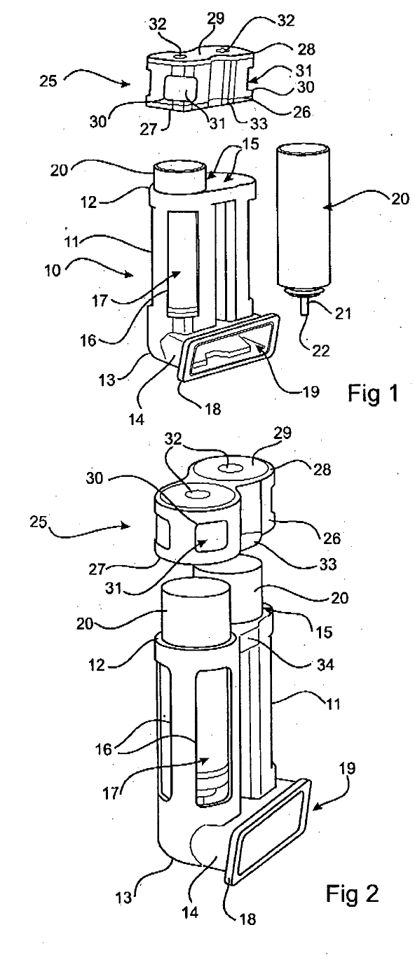

FIGS. 1 and 2 show a canister holder (10) and two cylindrical canisters (20) for the inhaler assembly (100) according to one embodiment of the present invention.

The canister holder (10) includes an elongated hollow holding portion (11) which has an open first end (12) and a closed second end (13) having an outlet formation (14) extending therefrom substantially perpendicularly to the holding portion (11).

The holding portion (11) includes two separate receiving portions (15) each having a substantially cylindrically shaped interior to receive a canister (20). The receiving portions (15) are joined to each other to hold the canisters (20) side by side and parallel to each other in use. The holding portions (11) include cut-out portions (16) therein which form windows (17) in order to easily identify the canisters (20) received in the canister holder (10).

The canisters (20) contain an aerosol propellant and medication to release a fixed dose of medication in aerosol form when actuated. Each canister (20) includes a metering dose valve (21) with an actuating stem (22). The closed second end (13) of the holding portion (11) holds a discharge nozzle for each canister (20) which mates with the respective metering valve (21) and actuating stem (22). When each canister (20) is actuated by depressing same towards the closed second end (13), the actuating stem (22) is moved inwardly into the canister (20) which allows the dosing valve (21) to release the aerosolised medication through the outlet formation (14). The outlet formation (14) is substantially rectangular in shape and includes a medication outlet (19), and a peripheral flange (18) extending outwardly around the periphery of the outlet (19).

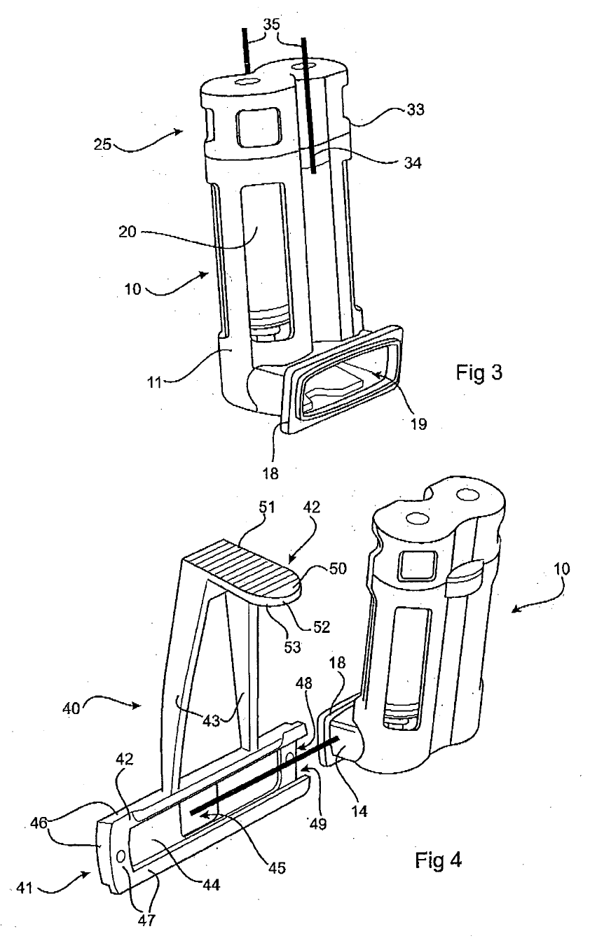

The canister holder (10) also includes a closing cap (25) which includes a hollow body (26) which is open at its first end (27) and substantially closed at its second end (28) via an end face (29). The cap body (26) substantially corresponds in cross-sectional shape to the holding portion (11), and also includes cutout portions (30) therein which act as windows (31). The end face (29) includes an actuator access aperture (32) for each canister (20). Formed adjacent the cap first end (27) is a flange (33) which corresponds to a flange (34) formed adjacent the holding portion first end (12). As shown in FIG. 3, the closing cap (25) is placed over the canisters (20) received in the holding portion (11) and the flanges (33) and (34) are fastened to each other as indicated at (35) to retain the canisters (20) in the canister holder (10). The flanges (33) and (34) can be fastened by screws or detachable attachment means.

FIG. 4 shows the assembled canister holder (10) of FIG. 3 being attached to an actuator (40). The actuator (40) includes a retaining portion (41) for receiving and retaining the canister holder outlet formation (14) and an actuator arm (42) attached to the retaining portion (41) by connectors (43). The retaining portion (41) has a rectangular shape substantially corresponding to that of the outlet formation (14) and includes a panel (44) having a substantially central opening (45) therein. First flanges (46) extend from the top, bottom and a first side edge of the panel (44), and second flanges (47) extend inwardly from the first flanges (46). The panel (44) and first and second flanges (46) and (47) together form a space (48) for slidably receiving and retaining the outlet formation flange (18) of the canister holder (10) which can be inserted therein via an open second side edge (49) of the retaining portion (41). When the outlet formation (14) is received in the retaining portion (41), the outlet (19) is substantially in line with the opening (45). The actuator arm (42) includes a first surface (50) having grip formations (51) for engagement by the fingers of a user, and an opposite second surface (52) from which extends a protrusion (53) which extends generally towards the retaining portion (41).

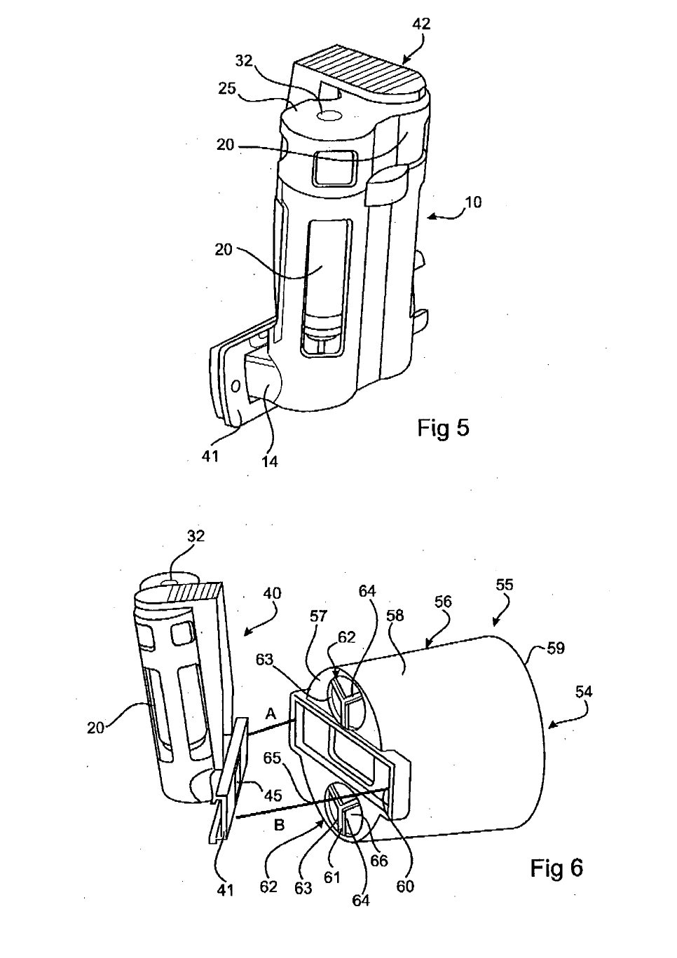

As shown in FIG. 5, when the outlet formation (14) is received by the retaining portion (41), the actuator arm (42) extends over the closing cap (25) and the protrusion (53) extends into one of the cap apertures (32) as desired. In use, the actuator arm (42) can be flexed generally toward the retaining portion (41) which presses the canisters (20) via the protrusion (53). This effectively depresses the canisters (20) in the holding portion (11) to release medication as desired through the outlet (19) and opening (45). The outlet formation (14) can be slid along the retaining portion (41) to align the protrusion (53) with either one of the apertures (32) and its corresponding canister (20).

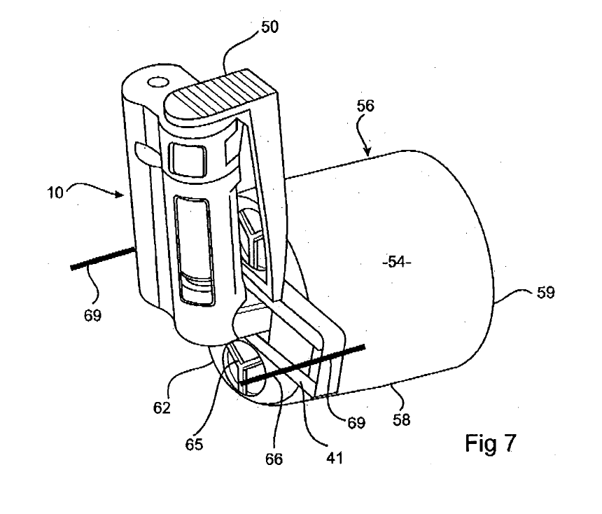

FIGS. 6 and 7 show a first half (56) of a spacer (55) for the inhaler assembly (100). The first half (56) is essentially a hollow cup having a substantially closed base (57), side walls (58) extending therefrom and an open end (59), together defining a chamber (54) therewithin. The base (57) includes a recess (60) which is adapted for releasably receiving and retaining the retaining portion (41) of the actuator (40). The recess (60) includes an aperture (61) centrally formed therein which corresponds to the aperture (45) in the retaining portion (41).

On either side of the recess (60) there are formed one-way valves (62). Each one-way valve (62) includes an opening (63) and three spokes (64) extending from the periphery of the opening (63) to meet at a centre (65). Attached to the centre 65 (adjacent the chamber (54)) is a flap (66) made of a flexible but resilient material which covers the opening (63). In use, negative pressure from within the chamber (54) forces the flaps (66) to flex inwardly, thus allowing air from outside the chamber (54) to enter same via the openings (63).

FIG. 7 shows the canister holder and actuator assembly attached to the spacer first half (56), where the recess aperture (61) is aligned with the aperture (45) in the retaining portion (41). The actuator retaining portion (41) is attached to the recess (60) via corresponding releasable formations or attachment means such as screws as indicated at (69).

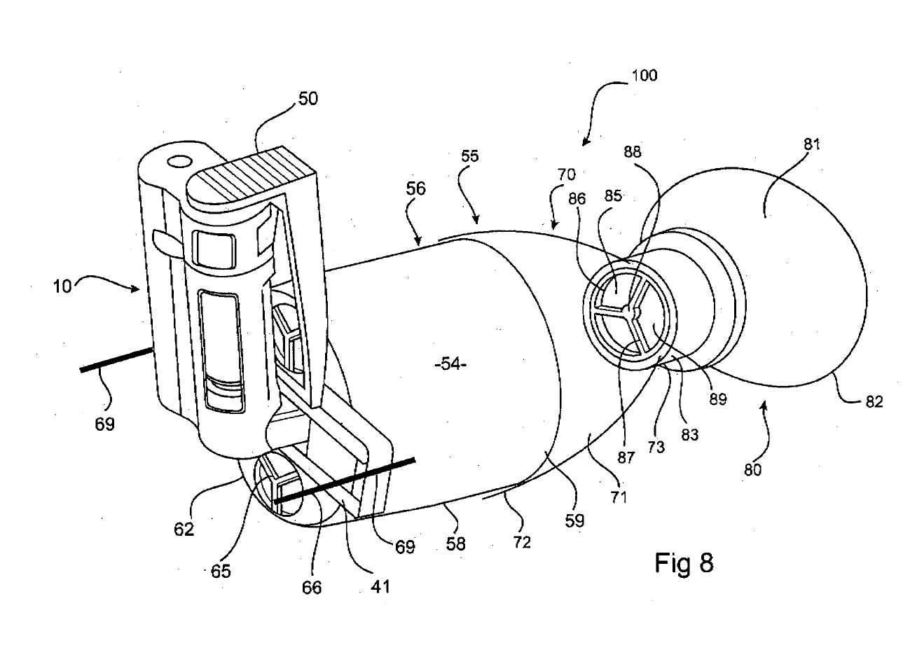

FIG. 8 shows the assembled inhaler assembly (100), where the spacer first half (56) is attached to a spacer second half (70) which is attached to a mask (80) for an animal such as a horse. The spacer second half (70) is hollow and substantially conical and includes a side wall (71), an open first end (72) adapted to attach to the first half open end (59) and an open second end (73) attached to the mask (80). The spacer first and second halves (56) and (70) together define the closed chamber (54) for the spacer (55), somewhat in the form of an expansion chamber.

The mask (80) includes a body (81) which is dimensioned and shaped to cover a single nostril of an animal as desired, such as a horse. The body (81) includes a first open end (82) shaped to substantially form a seal around the horse's nostril, and a second end (83) attached to the spacer second half second end (73).

The mask body (81) and the first and second ends (82) and (83) form a space (84) therewithin. A one-way valve (85) is formed at the mask second end (83) which is similar to the one way valves (62) and includes an opening (86), spokes (87) meeting at a centre (88), and a flap (89) attached to the centre (88) adjacent the mask space (84). In use, negative pressure from within the mask space (84) (such as when a horse inhales) forces the flap (89) to flex toward the space (84), thus allowing air and medication from within the chamber (54) to move into the mask space (84) for inhalation by the horse.

In use, the canister holder (10) is slidable along the actuator retaining portion (41), such that the actuator arm (50) can be positioned to actuate either the first or second canister (20) as desired. The first and second canisters (20) can comprise medication, which are to be administered in sequence or in combination with each other.

In sequence, the first medication canister (20) can be depressed to release the desired dose of first medication into the spacer chamber (54). The one-way valves (62) and (85) retain the aerosolised medication within the chamber (54). The mask (81) is then applied to the horse. The mask (80) covers a single nostril while the second nostril is manually occluded by the operator thereby forcing the animal to breathe through the nostril covered by the mask (80). Upon inhalation, negative pressure is produced within the mask space (84) and spacer chamber (54) which allows air from outside the spacer (55) to enter the chamber (54) via the one-way valves (62). This effectively pushes the aerosolised medication into the mask space (84) via the valve (85) which is inhaled by the horse.

If the horse does not inhale the entire dosage in one inhalation, it is substantially maintained within the chamber (54) and the medication can be inhaled by the horse upon its subsequent inhalation. After the first medication is inhaled, the canister holder (10) can be slid along the retaining portion (41) such that the second canister (20) can be actuated by the actuator arm (50). The second canister (20) is then actuated in a similar manner to release its medication into the chamber (54) for delivery to the horse.

If the medications in the first and second canisters (20) are designed to be combined with each other prior to delivery, both canisters (20) are actuated to deliver their medication into the chamber (54) prior to applying the mask (81) to the animal.

In cases where there is a need or desire to deliver more than two medications, the holder (10) as described above can be modified to include additional holding portions (11) for holding further canisters, such as adding a third canister (20) and the inhaler (100) can be operated in substantially the same manner as described above. This provides a suite of three different medications which can be delivered as a combined dose or in sequence using the same inhaler device once applied to the animal.

The present invention thus provides an inhaler assembly which provides effectively delivery of medication to an animal, including delivery of such medication in sequence or in combination with each other. It has been found, surprisingly, that the design of the inhaler assembly allows the delivery of more than one medication either in series or concomitantly without the need to change medications or inhaler devices, an aspect of particular relevance and importance in the treatment of respiratory disorders such as but not limited to heaves, Chronic Obstructive Pulmonary Disorder (COPD), Inflammatory airway Disease (IAD), Recurrent Airway Obstruction (RAO) or even forms of influenza.

Although a preferred embodiment of the present invention has been described it will be apparent to skilled persons that modifications can be made to the described embodiment or that the invention can be embodied in other forms. For example, the actuator arm (50) and the closing cap (25) can be omitted and the canisters (20) can be pressed individually as desired. The closing cap (25) can also be replaced with any suitable canister retaining means such as a flexible arm or the canisters (20) can have an interference fit with the dispensing valve at the holding portion second end. Also, the one way valve (85) can form part of the spacer (55) instead of the mask (80).

Throughout the specification and claims, unless the context requires otherwise, the word “comprise” or variations such as “comprises” or “comprising”, will be understood to imply the inclusion of a stated integer or group of integers but not the exclusion of any other integer or group of integers.

1. An inhaler assembly comprising a canister holder for retaining at least two canisters, the canister holder having an outlet in communication with the inlet of a spacer, the spacer having an outlet in communication with a mask wherein, in use, medication from at least one of the canisters can be dispensed into the spacer for inhalation via the mask.

2. The assembly as claimed in claim 1 wherein the holder is adapted to retain at least three diffusion canisters.

3. The assembly as claimed in claim 1, having at least two diffusion canisters received and retained therein.

4. (canceled)

5. The assembly as claimed in claim 1, wherein the canister holder includes an elongated holding portion having a separate receiving portion for receiving and retaining each canister.

6. The assembly as claimed in claim 5, wherein the holding portion includes an open first end and a closed second end having an outlet formation extending therefrom substantially perpendicularly to the holding portion.

7. The assembly as claimed in claim 5, wherein the receiving portions are joined to each other to hold the canisters side by side and substantially parallel to each other in use.

8. The assembly as claimed in claim 5 wherein the holding portions include cutout portions therein which form windows.

9. The assembly as claimed in claim 1, wherein each canister contains an aerosol propellant and medication to release a fixed dose of medication in aerosol form when actuated.

10. The assembly as claimed in claim 9, wherein each canister preferably includes a metering dose valve with an actuating stem.

11. The assembly as claimed in claim 6, wherein the closed second end of the holding portion holds a discharge nozzle for each canister which mates with the respective metering valve and actuating stem for releasing aerosolised medication through an outlet of the outlet formation.

12. The assembly as claimed in claim 6, wherein the canister holder includes a closing cap for placing over the canisters in the holding portion, the closing cap having an end face having an actuator access aperture for each canister.

13. The assembly as claimed in claim 1, wherein the canister holder is attachable to an actuator having an actuating arm for actuating the canisters.

14. The assembly as claimed in claim 13, wherein the actuator includes a retaining portion for receiving and retaining the canister holder outlet formation and wherein the actuating arm is attached to the retaining portion by connectors.

15. The assembly as claimed in claim 14, wherein the retaining portion includes a panel having a substantially central opening and a flange formation adapted for slidably receiving and retaining the outlet formation of the canister holder, wherein the outlet formation outlet is substantially in line with the panel opening.

16. The assembly as claimed in claim 13, wherein the actuating arm includes a first surface having grip formations and an opposed second surface from which a protrusion extends generally towards the retaining portion wherein, in use, the actuating arm extends over the closing cap and the protrusion extends into one of the cap apertures.

17. The assembly as claimed in claim 1, wherein the spacer includes a substantially closed first end face, side walls extending therefrom, and an open second end which together define a chamber therewithin, wherein the first end face includes an opening in communication with the canister holder outlet and the second end is in communication with the mask.

18. The assembly as claimed in claim 17, wherein the first end face of the spacer includes a recess which is adapted for receiving and retaining the actuator, wherein the recess includes an aperture centrally formed therein which corresponds to the aperture in the retaining portion.

19. The assembly as claimed in claim 17, wherein the first end face includes at least one one-way valve for allowing air into the chamber but substantially preventing the exit of air from the chamber.

20. The assembly as claimed in claim 19, wherein each one-way valve includes an opening which is closable by a flap made from a flexible but resilient material.

21. The assembly as claimed in claim 1, wherein the mask includes a body dimensioned and shaped to cover at least a single nostril of the nose of an animal.

22. The assembly as claimed in claim 21, wherein the body includes a first open end shaped to substantially form a seal around the animal's nostril, and a second end attached to the spacer second end.

23. The assembly as claimed in claim 21, wherein the mask is adapted to the body dimensions of any large animal selected from the group consisting of horses, cattle, sheep, camels, donkeys, ponies; small animals selected from the group consisting of cats, dogs and birds; and humans, both adult and children.

24. The assembly as claimed in claim 1, which includes a one-way valve between the spacer and the mask.

25. (canceled)

26. The assembly as claimed in claim 1, wherein the medication is selected from the group consisting of bronchodilators that facilitate the relaxation of the trachea through which humans and animals breathe, corticosteroids that reduce the inflammatory reaction to airway disorders, anti-mucolytic agents such as cromoglycate, antibiotics that reduce the microbial load associated with an infection and other forms of medication aimed at enhancing the quality of breathing or reducing in the short or longer term the severity of the disorder, and combinations thereof

27. A method of administering at least two types of inhalation medication in sequence to an animal using an inhaler assembly as claimed in claim 1, the method comprising the steps of:
depressing a first medication canister to release the desired dose of first medication into the spacer chamber;
applying the mask to cover a single nostril of the animal while the second nostril is manually occluded by the operator thereby forcing the animal to breathe through the nostril covered by the mask such that aerosolised medication in the chamber is inhaled by the animal; and
repeating the above steps with the remaining medication canister or canisters as required.

28. A method of administering at least two types of inhalation medication in combination to an animal using an inhaler assembly as claimed in claim 1, the method comprising:
depressing the medication canisters to release the desired dose of medication from each canister into the spacer chamber; and
applying the mask to cover a single nostril of the animal while the second nostril is manually occluded by the operator thereby forcing the animal to breathe through the nostril covered by the mask such that aerosolised combination of medication in the chamber is inhaled by the animal.

29-33. (canceled)
